(19) United States
(12) Patent Application Publication
(10) Pub. No.: US 2003/0216449 A1
(43) Pub. Date: Nov. 20, 2003

(54) Phosphate transport inhibitors

(76) Inventors: Joseph Weinstock, King of Prussia, PA (US); Gerald Girard, King of Prussia, PA (US); Dimitri Gaitanopoulos, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/275,661

(22) PCT Filed: May 11, 2001
(86) PCT No.: PCT/US01/15324

(51) Int. Cl. A61K 31/433; A61K 31/42; A61K 31/381; A61K 31/277; A61K 31/18

(52) U.S. Cl. 514/361; 514/438; 514/522; 514/602; 514/380

(57) ABSTRACT

N-Aryl-2-sulfonamidobenzamides, useful for treatment of chronic renal failure and uremic bone disease, are disclosed.

### FIELD OF THE INVENTION

The present invention involves the treatment of chronic renal failure, uremic bone disease and related diseases by inhibition of phosphate retention by certain N-aryl-2-sulfonamidobenzamides.

### BACKGROUND OF THE INVENTION

When kidneys are injured, the adaptive mechanisms involved in restoring homeostasis can lead to additional injury and an inexorable progression to end stage renal disease (ESRD) (Hostetter et al, Am. J. Physiol. 241:F85-F93 (1981)). ESRD affects more than 270,000 patients in the US. While the use of dialysis and kidney transplantation have dramatically improved the survival rate of patients with ESRD, a number of problems have appeared in these patients which complicates their long term management. Early and major contributors to the morbidity of patients with ESRD are abnormalities in mineral and bone metabolism induced by a progressive loss of renal excretory function. Among other factors, phosphate (Pi) retention has been identified as playing a major role in the progression of renal failure and in the generation of secondary hyperparathyroidism (HPTH) and uremic bone disease.

Evidence implicating a role for Pi retention in the progression of chronic renal failure (CRF) has come mainly from studies on experimental animals. Ibels et al, N. Engl. J. Med. 298:122-126, (1978), first demonstrated in a rat model of CRF that dietary Pi restriction prevented renal functional deterioration as assessed by stabilization or improvement of serum creatinine levels, reduced proteinuria, improved histology and reduced mortality. Similar findings were obtained in a rat model of nephrotoxic serum nephritis (Karlinsky et al, Kidney Int. 17:293-302 (1980)). However, these studies were criticized on the basis that a low Pi diet is associated with decreased food intake and thus protein intake which by itself can reduce the progression of CRF. Therefore, Lumlertgul et al, Kidney Int. 29:658-666, (1986) placed 5/6th nephrectomized rats on a normal Pi diet but gave one group a Pi binder. All rats were pair fed and had similar caloric, protein, carbohydrate, vitamin and mineral intakes. At both 6 and 12 weeks rats ingesting the Pi binder showed a lower protein excretion, lower serum creatinine level, lower renal calcium content and less histologic scarring than rats not receiving the Pi binder. This study demonstrated unequivocally that dietary Pi restriction can have beneficial effects on the progression of CRF independent of caloric and protein intake in experimental animals.

In addition to the beneficial effects of dietary Pi restriction on the progression of CRF discussed above, evidence has also been found that dietary Pi excess can accelerate the progression of CRF. A number of studies in rat models of CRF (Kleinknecht et al, Kidney Int. 5:534-541, (1979); Haut et al, Kidney Int. 17:722-731, (1980); Gimenez et al, Kidney Int. 22:36-41, (1982)) have shown that diets high in Pi lead to a more rapid deterioration in renal function as assessed by serum creatinine levels and the severity of histologic lesions.

Some evidence also suggests that dietary Pi restriction may slow the progression of CRF in patients. Maschio et al, Kidney Int., 22:371-376, (1982) and Maschio et al, Kidney Int., 24:S 273-S 277, (1983) placed patients with mild or moderate renal insufficiency on diets restricted in protein and Pi for up to 76 months. They found that the rate of decline in renal function was slower in the dietary restricted group than in the control group, especially in patients with mild CRF. Barsotti et al., Kidney Int. 24:S278-S284, (1983) and Barsotti et al., Clin. Nephrol. 21:54-59, (1984) placed CRF patients on either a low protein diet or on a low protein-low Pi diet and found that the rate of decline in renal function slowed after the institution of dietary restrictions in both groups. Importantly, they also observed a slower rate of decline in patients on the low protein-low Pi diet compared to the low protein diet alone. In a study of 4 children placed on a low Pi diet serum creatinine levels were halved during the 6 months on the restricted diet compared with a similar period on a normal diet (McCrory et al, J. Pediatr. 111:410-412, (1987). Furthermore, growth velocity in these children increased significantly on the low Pi diet compared with the control period. Other human studies (Barrientos et al, Electrolyte Metab. 7:127-133, (1982); Ciadrella et al, Nephron 42:196-199, (1986); Gin et al, Metabolism 36:1080-1085, (1987)), mainly of short duration, have failed to observe an effect of Pi restriction on the course of CRF. Nevertheless, the bulk of the animal studies discussed above together with the less well controlled human studies suggest that dietary restriction of Pi is beneficial in slowing the progression of CRF, especially in mild to moderate renal insufficiency.

The mechanism by which Pi excess leads to an increase in the rate of renal failure is unknown. However, most evidence supports an interaction between Pi and cellular Ca²⁺ accumulation. In the failing kidney a rise in the filtered load of Pi together with a reduction in Pi reabsorption secondary to elevated levels of parathyroid hormone (PTH) results in an increase in tubular fluid Pi concentration. This leads to an increased transepithelial flux of Ca²⁺ and elevated cellular Ca²⁺ levels resulting in Ca²⁺-induced cell injury (Borle et al., Endocrinology 102:1725-1732, (1978). Alternatively, or in addition, calcium-phosphate precipitation may occur resulting in renal calcification and nephrocalcinosis (Lau, K., Kidney Int. 36:918-937, (1989)).

Finally, Shapiro et al., Am. J. Physiol. 258:F183-F188, (1990) suggested that the renal hypermetabolism normally associated with the 5/6th nephrectomized model of CRF in rats may contribute to the progression of CRF in this model. Thus, restriction of dietary Pi reduced renal oxygen consumption by 50% and reduced intracelluar Pi concentrations without altering the steady state concentration of ATP as assessed by ³¹P-NMR in this model.

Chronic renal failure (CRF) affects more than 270,000 patients in the US alone and costs an estimated $6.8 billion in annual heath care costs. Early and major contributors to the morbidity of CRF patients are abnormalities in electrolyte and bone metabolism induced by the progressive loss of renal excretory function. Phosphate (Pi) retention has been identified as playing a major role in the progression of CRF and in the development of uremic bone disease.

Studies in the literature have shown that dietary Pi restriction slows the progression of CRF in animal models and in small patient studies; decreases elevated plasma PTH levels in CRF animal models and patients; and increases the circulating levels of 1, 25 (OH)₂ vitamin D and intestinal Ca²⁺ absorption.

Thus, inhibition of Pi transport by the gut and kidney is considered beneficial in slowing the progression of CRF and uremic bone disease. Thus, inhibition of Pi transport by the gut and kidney is beneficial in slowing the progression of CRF and uremic bone disease.

Consequently, there exists a need to find an alternative means of reducing phosphate retention, in mammals, in addition to diet restriction of phosphate for the treatment of renal diseases, and uremic bone disease.

### SUMMARY OF THE INVENTION

The present invention involves novel methods of using of N-aryl-2-sulfonamidobenzamides as phosphate transport inhibitors for the selective inhibition of Pi transport in the kidney and/or the intestine as a therapeutic treatment in chronic renal failure and uremic bone disease.

### DETAILED DESCRIPTION OF THE INVENTION

The present invention involves the use of inhibitors of phosphate transport, for the treatment of chronic renal failure, and uremic bone disease, as well as other related diseases, such as hyperphosphatemia, vitamin D metabolism, and secondary hyperparathyroidism caused by the retention of phosphate. Preferably, inhibitors for use herein are those which selectively inhibit Na⁺-dependent Pi transport in tissues, preferably renal and intestinal tissue, from a number of species, including human.

The present invention relates to the use of compounds that are inhibitors of sodium-dependent phosphate transport, which are represented by the following Formula (I):

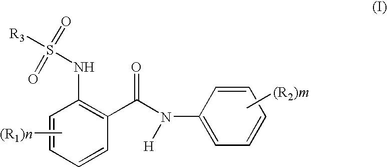

wherein:

R₁ and R₂ are independently selected from the group consisting of hydrogen, alkyl, alkenyl, arylalkyl, acyl, aroyl, haloalkyl, aryl, heteroaryl, halo, carboxy, carboalkoxy, carbamyl, alkylcarbamyl, arylcarbamyl, cyano, alkoxy, hydroxyl, phenylazo, amino, nitro, alkylamino, arylamino, arylalkylamino, acylamino, aroylamino, alkylthio, arylalkylthio, arylthio, alkysulfinyl, arylsulfinyl, arylalkylsulfinyl, alkylsulfonyl, arylsulfonyl, arylalkylsulfonyl, sulfamyl, arylsulfonamido, and alkylsulfonamido;

or the R₁ and or the R₂ moiety represents a fusing element forming a benzothiophene, naphthalene, quinoline, or isoquinoline with the ring it substitutes;

or (R₁)ₙ and or (R₂)m and the ring it substitutes represents a heterocycle selected from the group consisting of thiophene, furan, pyridine, pyrimidine, and pyrazine, and benzo analogs thereof; and

R₃ is independently selected from the group consisting of alkyl, haloalkyl, R₁ aryl and R₁ aralkyl, and R₁ substituted heterocycles selected from the group consisting of thiophene, furan, pyridine, pyrimidine, pyrazine, imidazole, and thiazole, isoxazole, thiadiazole, oxadiazole, and benzo analogs thereof.

As used herein, “alkyl” refers to an optionally substituted hydrocarbon group joined together by single carbon-carbon bonds. Preferred alkyl substituents are as indicated throughout. The alkyl hydrocarbon group may be linear, branched or cyclic, saturated or unsaturated.

As used herein, “aryl” refers to an optionally substituted aromatic group with at least one ring having a conjugated pi-electron system, containing up to two conjugated or fused ring systems. “Aryl” includes carbocyclic aryl, heterocyclic aryl and biaryl groups, all of which may be optionally substituted. Preferred aryl substituents are as indicated throughout.

The compounds of the present invention may contain one or more asymmetric carbon atoms and may exist in racemic and optically active forms. All of these compounds and diastereomers are contemplated to be within the scope of the present invention.

Preferred compounds include, but are not limited to:

N-phenyl-2-(3-trifluoromethylphenylsulfonamido)benzamide;

5-Methoxy-N-(3-trifluoromethylphenyl)-2-(4-chlorophenylsulfonamido)benzamide;

5-Bromo-N-(4-Bromophenyl)-2-(5-chloro-2-thienylsulfonamido)benzamide;

5-Bromo-N-(4-Bromophenyl)-2-(3,3,3-trifluoroethylsulfonamido)benzamide;

5-Bromo-N-(4-Bromophenyl)-2-(3-chloro-2-fluorophenylsulfonamido)benzamide;

5-Bromo-N-(4-Bromophenyl)-2-(3-chloropropylsulfonamido)benzamide;

5-Bromo-N-(4-Bromophenyl)-2-(4-methoxyphenylsulfonamido)benzamide;

5-Bromo-N-(4-Bromophenyl)-2-(2-fluorophenylsulfonamido)benzamide;

N-(4-Chlorophenyl)-2-(2-fluorophenylsulfonamido)benzamide;

N-(4-Bromophenyl)-2-(3,3,3-trifluoroethylsulfonamido)benzamide;

N-(4-Bromophenyl)-5-chloro-2-(3-chloro-2-fluorophenylsulfonamido)benzamide;

N-(4-Chlorophenyl)-2-(3,4-dichlorophenylsulfonamido)benzamide;

N-(4-Bromophenyl)-2-(2-thienylsulfonamido)benzamide;

N-(4-Bromophenyl)-2-(2-methoxycarbonyl-3-thienylsulfonamido)benzamide;

N-(3,4-Dichlorophenyl)-2-(2-fluorophenylsulfonamido)benzamide;

N-(4-Chlorophenyl)-2-(3-trifluoromethylphenylsulfonamido)benzamide;

5-Bromo-N-(4-chlorophenyl)-2-(3,4-dichlorophenylsulfonamido)benzamide;

N-(4-chlorophenyl)-2-(3,4-difluorophenylsulfonamido)benzamide;

N-(4-Butoxyphenyl)-2-(3,4-difluorophenylsulfonamido)benzamide;

N-(4-Chlorophenyl)-2-(3,5-dimethylisoxazole-4-sulfonamido)benzamide;

N-(4-Chlorophenyl)-2-(2,1,3-benzothiadiazole-4-sulfonylamino)-benzamide;

N-(3-Trifluoromethoxyphenyl)-2-(5-Bromo-thiophene-2-sulfonylamino)-benzamide;

N-(4-Bromophenyl)-2-(phenylsulfonamido)benzamide and

5-Methoxy-N-(4-chlorophenyl)-2-(3-trifluoromethylphenylsulfonamido)benzamide.

Pharmaceutically acceptable salts for use when basic groups are present include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present.

The present invention provides compounds of Formula (I) above which can be prepared using standard techniques. An overall strategy for preparing preferred compounds described herein can be carried out as described in this section. Using the protocols described herein as a model, one of ordinary skill in the art can readily produce other compounds of the present invention.

With appropriate manipulation and protection of any chemical functionality, synthesis of the remaining compounds of Formula (I) is accomplished by methods analogous to those above and to those described in the Experimental section.

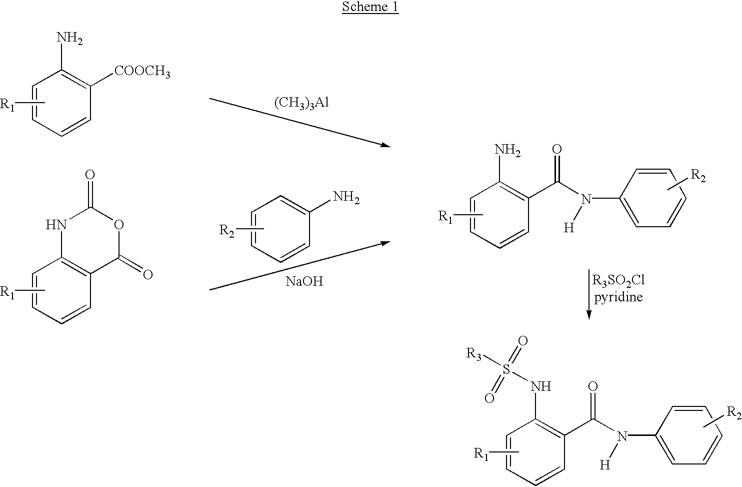

In order to use a compound of Formula (I) or a pharmaceutically acceptable salt thereof for the treatment of humans and other mammals, it is normally formulated in accordance with standard pharmaceutical practice as a pharmaceutical composition.

The present compounds can be administered by different routes including intravenous, intraperitoneal, subcutaneous, intramuscular, oral, topical (transdermal), or transmucosal administration. For systemic administration, oral administration is preferred. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and re-dissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various compounds to be administered can be determined by standard procedures taking into account factors such as the compound IC₅₀, EC₅₀, the biological half-life of the compound, the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula (I). A topical formulation contains suitably 0.01 to 5.0% of a compound of Formula (I). The active ingredient may be administered from 1 to 6 times per day, preferably once, sufficient to exhibit the desired activity, as is readily apparent to one skilled in the art.

As used herein, “treatment” of a disease includes, but is not limited to prevention, retardation and prophylaxis of the disease.

Composition of Formula (I) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil. olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

No unacceptable toxological effects are expected when compounds of the present invention are administered in accordance with the present invention.

Sodium-dependent phosphate transport inhibition is determined by the ability of the test compound to inhibit the uptake of radio-labeled inorganic phosphate by proximal tubule cells. Appropriate cells from human, rabbit, or rat may be used.

Cell Preparation and Phosphate Uptake Assay.

Rabbit proximal tubule cells were isolated and cultured according to the procedure of Sakhrani, L. M. et al., Am. J. Physiol. 246: F757-F764, (1984) whose disclosure is incorporated herein by reference in its entirety. Human proximal tubule cells were purchased from Clonetics (San Diego, Calif.) and grown according to the suppliers' instructions. On the day of the experiment, cells were harvested from culture plates with 0.5 mM EDTA in phosphate buffered saline. The cells were washed twice in uptake buffer (see below) and equilibrated at 37 C. in the same buffer for 30 minutes. Aliquots of cells (100 ul, 0.5 to 1 million cells) were distributed into glass test tubes. Fifty ul of drug solution or buffer were added followed by 50 ul of uptake buffer containing 100 uM [³²P]-K₂HPO₄ (0.5 to 1 uCi/tube). After varying periods of time (usually 4 minutes) at 37 C., uptakes were stopped with 4 ml of cold stop solution (see below) and the cells were washed 3 times in this solution by centrifugation. The pelleted cells were dissolved in 0.5 ml 1N NaOH and ³²P was counted in a liquid scintillation counter. Phosphate uptake is expressed as pmol phosphate/mg cell protein.

| Stop solution | Uptake Buffer pH 7.4 |
|---|---|
| Mannitol 100 mM | NaCl 143 mM |
| NaCl 100 mM | Hepes 15 mM |
| Na Arsenate 10 mM | KCl 5.4 mM |
| Hepes 5 mM | MgCl₂ 0.8 mM |
| | CaCl₂ 1.8 mM |
| | Glucose 0.1% |

In the above noted whole cell assay system for rabbit and human proximal tubule cells the cells are harvested by filtration and ³²P uptake is measured. It is also possible to use ³³P rather than ³²P. Using human proximal tubule cells the IC₅₀ for 5-bromo-N-(4-bromophenyl)-2-(5-chloro-2-thienylsulfonamido)benzamide, 5-bromo-N-(4-bromophenyl)-2-(2-fluorophenylsulfonamido)benzamide, and 5-bromo-N-(4-bromophenyl)-2-(3-chloropropylsulfonamido)benzamide are 12, 15, and 14 μM respectively.

The following examples illustrate preparation of compounds and pharmaceutical compositions that may be used in this invention. The examples are not intended to limit the scope of this invention as defined hereinabove and as claimed below.

### EXAMPLE 1

N-(4-Bromophenyl)-2-amino-5-bromobenzamide

A 11.6 ml portion of a 2.0 M solution of trimethylaluminum (23.2 mmol) was added to a solution of 4.0 g (23.25 mmol) of 4-bromoaniline at 0° C. The reaction mixture was held at ambient temperature for 45 min, and then cooled to 0° C. Methyl 2-amino-5-bromobenzoate (4.72 g, 23.25 mmol) was added in small portions, and after a vigorous gas evolution ceased the reaction mixture was held at ambient temperature for 18 hr. The reaction mixture was then poured into 250 ml of 10% HCl (further gas evolution occurred) and the solid, which formed, collected by filtration. The solid was washed in turn with water and toluene and then dried at room temperature. TLC silica, CHCl₃:MeOH 9:1 with a drop of formic acid, R_{f} 0.80-0.90 and NMR identical with that of an authentic sample. This is a general procedure, which works with a wide variety of aromatic and heteroaromatic anthranilic acid and aniline analogs.

A mixture of 12.1 g (50 mmol) of 5-bromoisatoic anhydride, 9.4 g (55 mmol) of 4-bromoaniline, and 0.2 g (5 mmol) of NaOH in 150 ml of dioxane was refluxed for 18 hr. The cooled reaction mixture was filtered and concentrated under vacuum. The residue crystallized on addition of 95% EtOH. The solid was collected by filtration and washed with ethanol. A sample purified by thick layer chromatography (silica, 15% EtOAc in hexane) gave the expected NMR, MS, and elemental analysis.

A similar procedure starting from 5-chloroisatoic anhydride and 4-bromoaniline gave N-(4-bromophenyl)-2-amino-5-chlorobenzamide which gave the expected NMR, MS, and elemental analysis.

### EXAMPLE 2

5-Bromo-N-(4-Bromophenyl)-2-(4-chlorophenylsulfonylamino)benzamide

A solution of N-(4-Bromophenyl)-2-amino-5-bromobenzamide (8.64 g, 23.3 mmol), 4-chlorobenzenesulfonyl chloride (4.98 g, 23.6 mmol), and 7.37 g (93.2 mmol) in 300 ml of CH₂Cl₂ was allowed to stand at room temperature for 2 days. The reaction mixture was concentrated under vacuum and the residue dissolved in EtOAc. The solution was washed twice with 10% HCl, water, 5% NaHCO₃, water, and dried over MgSO₄. Concentration and recrystallization from 10% EtOAc in hexane gave product which had satisfactory NMR, MS, and elemental analysis.

### EXAMPLE 3

5-Bromo-N-(4-Bromophenyl)-2-(4-bromophenylsulfonylamino)benzamide

A solution of 31.5 mg (85 μmol) of N-(4-Bromophenyl)-2-amino-5-bromobenzamide, 32.5 mg (127.5 μmol) of 4-bromobenzenesulfonyl chloride, and 28 μl (340 μmol) of pyridine in 1 ml of CH₂Cl₂ was agitated for 18 hr. Then 84.5 mg (382 μmol) of polyamine resin HL (Nova Biochem, 4.53 mmol/g) was added, the mixture agitated for 18 hr, and the solids removed by filtration. Concentration under vacuum and purification by preparative HPLC (C18, 20-95% acetonitrile-0.1% aqueous TFA) gave product which gave a satisfactory HPLC-MS analysis.

Using procedures similar to those in Examples 2 and 3, the products from reaction of 5-bromo-N-(4-bromophenyl)-2-(4-chlorophenylsulfonylamino) benzamide with the following sulfonyl chlorides were obtained: 3-chlorophenyl-, 4-chlorophenyl-, 3,4-dichlorophenyl-, 3-chloro-4-fluoro-, 2-fluorophenyl-, 2,5-dimethoxyphenyl-, 3,4-dimethoxyphenyl-, 4-n-butoxyphenyl-, 2-trifluoromethylphenyl-, 4-phenylazophenyl-, 4-trifluoromethylphenyl-, 3,5-bis-trifluoromethylphenyl-, 2-methylphenyl-, 2,4,6-trimethylphenyl-, 2-naphthyl-,methane-, trifluoromethane-, 2-thienyl-, 5-chloro-2-thienyl-, 4-biphenylyl-, 3-chloropropyl-, 4-cyanophenyl-, 3,5-dichlorophenyl-, styryl-, 2-methoxycarbonyl-3-thienyl-, 4-iodophenyl-, 2,6-dichlorophenyl-, 4-t-butylphenyl-, and 2,2,2-trifluoroethyl-. The products gave satisfactory results on HPLC-MS analyses.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A method of inhibiting sodium-dependent phosphate transport by administering to a subject in need thereof a safe and effective amount of a compound according to Formula (I): 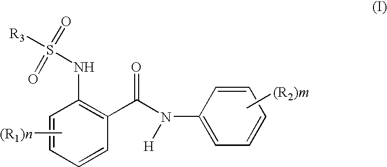
wherein:
R₁ and R₂ are independently selected from the group consisting of hydrogen, alkyl, alkenyl, arylalkyl, acyl, aroyl, haloalkyl, halo, carboxy, carboalkoxy, carbamyl, alkylcarbamyl, arylcarbamyl, cyano, alkoxy, hydroxyl, phenylazo, amino, nitro, alkylamino, arylamino, arylalklylamino, acylamino, aroylamino, alkylthio, arylalkylthio, arylthio, alkysulfinyl, arylsulfinyl, arylalkylsulfinyl, alkylsulfonyl, arylsulfonyl, arylalkylsulfonyl, sulfamyl, arylsulfonamido, and alkylsulfonamido;
or the R₁ moiety represents a fused ring forming a benzothiophene, naphthalene, quinoline, or isoquinoline with the ring it substitutes;
or (R₁)ₙ and the ring it substitutes represents a heterocycle selected from the group consisting of thiophene, furan, pyridine, pyrimidine, and pyrazine, and benzo analogs thereof; and
R₃ is independently selected from the group consisting of alkyl, haloalkyl, R₁ aryl and R₁ aralkyl, R₁ benzoaryl and R₁ benzoaralkyl, and R₁substituted heterocycles selected from the group consisting of thiophene, furan, pyridine, pyrimidine, pyrazine, imidazole, isoxazole, thiadiazole, oxadiazole, and thiazole, and benzo analogs thereof.

2. The method according to claim 1 wherein the compound is selected from the group consisting of:
N-phenyl-2-(3-trifluoromethylphenylsulfonamido)benzamide;
5-Methoxy-N-(3-trifluoromethylphenyl)-2-(4-chlorophenylsulfonamido)benzamide;
5-Bromo-N-(4-Bromophenyl)-2-(5-chloro-2-thienylsulfonamido)benzamide;
5-Bromo-N-(4-Bromophenyl)-2-(3,3,3-trifluoroethylsulfonamido)benzamide;
5-Bromo-N-(4-Bromophenyl)-2-(3-chloro-2-fluorophenylsulfonamido)benzamide;
5-Bromo-N-(4-Bromophenyl)-2-(3-chloropropylsulfonamido)benzamide;
5-Bromo-N-(4-Bromophenyl)-2-(4-methoxyphenylsulfonamido)benzamide;
5-Bromo-N-(4-Bromophenyl)-2-(2-fluorophenylsulfonamido)benzamide;
N-(4-Chlorophenyl)-2-(2-fluorophenylsulfonamido)benzamide;
N-(4-Bromophenyl)-2-(3,3,3-trifluoroethylsulfonamido)benzamide;
N-(4-Bromophenyl)-5-chloro-2-(3-chloro-2-fluorophenylsulfonamido)benzamide;
N-(4-Chlorophenyl)-2-(3,4-dichlorophenylsulfonamido)benzamide;
N-(4-Bromophenyl)-2-(2-thienylsulfonamido)benzamide;
N-(4-Bromophenyl)-2-(2-methoxycarbonyl-3-thienylsulfonamido)benzamide;
N-(3,4-Dichlorophenyl)-2-(2-fluorophenylsulfonamido)benzamide;
N-(4-Chlorophenyl)-2-(3-trifluoromethylphenylsulfonamido)benzamide;
5-Bromo-N-(4-chlorophenyl)-2-(3,4-dichlorophenylsulfonamido)benzamide;
N-(4-chlorophenyl)-2-(3,4-difluorophenylsulfonamido)benzamide;
N-(4-Butoxyphenyl)-2-(3,4-difluorophenylsulfonamido)benzamide;
N-(4-Chlorophenyl)-2-(3,5-dimethylisoxazole-4-sulfonamido)benzamide;
N-(4-Chlorophenyl)-2-(2,1,3-benzothiadiazole-4-sulfonylamino)-benzamide;
N-(3-Trifluoromethoxyphenyl)-2-(5-Bromo-thiophene-2-sulfonylamino)-benzamide;
N-(4-Bromophenyl)-2-(phenylsulfonamido)benzamide and
5-Methoxy-N-(4-chlorophenyl)-2-(3-trifluoromethylphenylsulfonamido)benzamide.

3. A method of causing phosphate excretion and/or inhibiting phosphate absorption by administering to a subject in need thereof a safe and effective amount of a compound according to Formula (I): 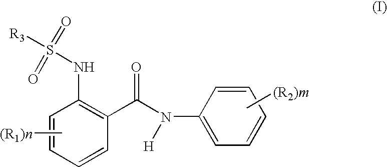
wherein:
R₁ and R₂ are independently selected from the group consisting of hydrogen, alkyl, alkenyl, arylalkyl, acyl, aroyl, haloalkyl, halo, carboxy, carboalkoxy, carbamyl, alkylcarbamyl, arylcarbamyl, cyano, alkoxy, hydroxyl, phenylazo, amino, nitro, alkylamino, arylamino, arylalkylamino, acylamino, aroylamino, alkylthio, arylalkylthio, arylthio, alkysulfinyl, arylsulfinyl, arylalkylsulfinyl, alkylsulfonyl, arylsulfonyl, arylalkylsulfonyl, sulfamyl, arylsulfonamido, and alkylsulfonamido;
or the R₁ moiety represents a fused ring forming a benzothiophene, naphthalene, quinoline, or isoquinoline with the ring it substitutes;
or (R₁)ₙ and the ring it substitutes represents a heterocycle selected from the group consisting of thiophene, furan, pyridine, pyrimidine, and pyrazine, and benzo analogs thereof; and
R₃ is independently selected from the group consisting of alkyl, haloalkyl, R₁ aryl and R₁ aralkyl, and R₁ substituted heterocycles selected from the group consisting of thiophene, furan, pyridine, pyrimidine, pyrazine, imidazole, isoxazole, thiadiazole, oxadiazole and thiazole, and benzo analogs thereof.

4. A pharmaceutical composition comprising a compound of claim 1 and a pharmaceutically acceptable carrier.

5. A method of treating chronic renal failure by inhibiting the phosphate transport system in a mammal in need thereof, by administering to a subject in need thereof a safe and effective amount of a compound a compound according to Formula (I): 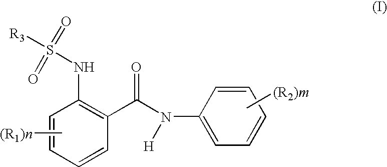
wherein:
R₁ and R₂ are independently selected from the group consisting of hydrogen, alkyl, alkenyl, arylalkyl, acyl, aroyl, haloalkyl, halo, carboxy, carboalkoxy, carbamyl, alkylcarbamyl, arylcarbamyl, cyano, alkoxy, hydroxyl, phenylazo, amino, nitro, alkylamino, arylamino, arylalkylamino, acylamino, aroylamino, alkylthio, arylalkylthio, arylthio, alkysulfinyl, arylsulfinyl, arylalkylsulfinyl, alkylsulfonyl, arylsulfonyl, arylalkylsulfonyl, sulfamyl, arylsulfonamido, and alkylsulfonamido;
or the R₁ moiety represents a fused ring forming a benzothiophene, naphthalene, quinoline, or isoquinoline with the ring it substitutes;
or (R₁)ₙ and the ring it substitutes represents a heterocycle selected from the group consisting of thiophene, furan, pyridine, pyrimidine, and pyrazine, and benzo analogs thereof; and
R₃ is independently selected from the group consisting of alkyl, haloalkyl, R₁ aryl and R₁ aralkyl, R₁ benzoaryl and R₁ benzoaralkyl, and R₁ substituted heterocycles selected from the group consisting of thiophene, furan, pyridine, pyrimidine, pyrazine, isoxazole, thiadiazole, oxadiazole, imidazole, and thiazole, and benzo analogs thereof.

6. A method according to claim 5 wherein uremic bone disease is treated.

7. A method according to claim 5 wherein the phosphate transport is inhibited in the kidney.

8. A method according to claim 5 wherein the phosphate transport is inhibited in the intestine.

9. A pharmaceutical composition comprising a compound selected from the group consisting of:
5-Bromo-N-(4-Bromophenyl)-2-(2-fluorophenylsulfonamido)benzamide;
5-Bromo-N-(4-Bromophenyl)-2-(5-chloro-2-thienylsulfonamido)benzamide;
5-Bromo-N-(4-Bromophenyl)-2-(3,3,3-trifluoroethylsulfonamido)benzamide;
5-Bromo-N-(4-Bromophenyl)-2-(3-chloro-2-fluorophenylsulfonamido)benzamide;
5-Bromo-N-(4-Bromophenyl)-2-(3-chloropropylsulfonamido)benzamide;
5-Bromo-N-(4-Bromophenyl)-2-(2-methylphenylsulfonamido)benzamide;
5-Bromo-N-(4-Bromophenyl)-2-(2-thienylsulfonamido)benzamide;
5-Bromo-N-(4-Bromophenyl)-2-(2-methoxycarbonyl-3-thienylsulfonamido)benzamide;
5-Bromo-N-(4-Bromophenyl)-2-(4-cyanophenylsulfonamido)benzamide;
5-Bromo-N-(4-Bromophenyl)-2-(methylsulfonamido)benzamide;
5-Bromo-N-(4-Bromophenyl)-2-(3-trifluoromethylphenylsulfonamido)benzamide;
5-Bromo-N-(4-Bromophenyl)-2-(4-methoxyphenylsulfonamido)benzamide;
5-Bromo-N-(4-Bromophenyl)-2-(phenylsulfonamido)benzamide and
5-Bromo-N-(4-Bromophenyl)-2-(4-bromophenylsulfonamido)benzamide; and a pharmaceutically acceptable carrier.
N-phenyl-2-(3-trifluoromethylphenylsulfonamido)benzamide;
5-Methoxy-N-(3-trifluoromethylphenyl)-2-(4-chlorophenylsulfonamido)benzamide;
5-Bromo-N-(4-Bromophenyl)-2-(5-chloro-2-thienylsulfonamido)benzamide;
5-Bromo-N-(4-Bromophenyl)-2-(3,3,3-trifluoroethylsulfonamido)benzamide;
5-Bromo-N-(4-Bromophenyl)-2-(3-chloro-2-fluorophenylsulfonamido)benzamide;
5-Bromo-N-(4-Bromophenyl)-2-(3-chloropropylsulfonamido)benzamide;
5-Bromo-N-(4-Bromophenyl)-2-(4-methoxyphenylsulfonamido)benzamide;
5-Bromo-N-(4-Bromophenyl)-2-(2-fluorophenylsulfonamido)benzamide;
N-(4-Chlorophenyl)-2-(2-fluorophenylsulfonamido)benzamide;
N-(4-Bromophenyl)-2-(3,3,3-trifluoroethylsulfonamido)benzamide;
N-(4-Bromophenyl)-5-chloro-2-(3-chloro-2-fluorophenylsulfonamido)benzamide;
N-(4-Chlorophenyl)-2-(3,4-dichlorophenylsulfonamido)benzamide;
N-(4-Bromophenyl)-2-(2-thienylsulfonamido)benzamide;
N-(4-Bromophenyl)-2-(2-methoxycarbonyl-3-thienylsulfonamido)benzamide;
N-(3,4-Dichlorophenyl)-2-(2-fluorophenylsulfonamido)benzamide;
N-(4-Chlorophenyl)-2-(3-trifluoromethylphenylsulfonamido)benzamide;
5-Bromo-N-(4-chlorophenyl)-2-(3,4-dichlorophenylsulfonamido)benzamide;
N-(4-chlorophenyl)-2-(3,4-difluorophenylsulfonamido)benzamide;
N-(4-Butoxyphenyl)-2-(3,4-difluorophenylsulfonamido)benzamide;
N-(4-Chlorophenyl)-2-(3,5-dimethylisoxazole-4-sulfonamido)benzamide;
N-(4-Chlorophenyl)-2-(2,1,3-benzothiadiazole-4-sulfonylamino)-benzamide;
N-(3-Trifluoromethoxyphenyl)-2-(5-Bromo-thiophene-2-sulfonylamino)-benzamide;
N-(4-Bromophenyl)-2-(phenylsulfonamido)benzamide and
5-Methoxy-N-(4-chlorophenyl)-2-(3-trifluoromethylphenylsulfonamido)benzamide.
